(19) United States
(12) Patent Application Publication
(10) Pub. No.: US 2005/0065189 A1
(43) Pub. Date: Mar. 24, 2005

(54) Thiazole derivatives as cannabinoid receptor modulators

(75) Inventors: Josephus H.M. Lange, Weesp (NL); Cornelis G. Kruse, Weesp (NL); Herman H. Van Stuivenberg, Weesp (NL); Leonardus A.J.M. Sliedregt, Weesp (NL)

Correspondence Address: Finnegan, Henderson, Farabow,;Garrett & Dunner, L.L.P., 1300 I Street, N.W., Washington, DC, 20005-3315, US

(73) Assignee: SOLVAY PHARMACEUTICALS B.V.

(21) Appl. No.: 10/942,021

(22) Filed: Sep. 16, 2004

Related U.S. Application Data
(60) Provisional application No. 60/504,212, filed on Sep. 22, 2003.

(30) Foreign Application Priority Data
Sep. 19, 2003 (EP) 03078309.6

(51) Int. Cl. C07D 277/38; C07D 417/02; A61K 31/4439; A61K 31/427

(52) U.S. Cl. 514/342; 514/365; 548/200; 546/269.7

(57) ABSTRACT

The present invention relates to a group of thiazole derivatives, to methods for the preparation of these compounds, to pharmaceutical compositions containing at least one these compounds as active ingredient, as well to the use of these compositions for the treatment of psychiatric and neurological disorders and other diseases involving cannabinoid CB neurotransmission. The thiazole derivatives of the invention are either cannabinoid (CB) receptor antagonists, CB receptor agonists, CB receptor inverse agonists or CB receptor partial agonists.
The compounds have the general formula (I) 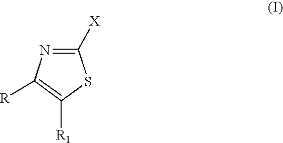
wherein R, R₁ and X have the meanings given in the specification.

The present invention relates to a group of thiazole derivatives, to methods for the preparation of these compounds, to pharmaceutical compositions containing at least one these compounds as active ingredient, as well to the use of these compositions for the treatment of psychiatric and neurological disorders and other diseases involving cannabinoid CB neurotransmission. The thiazole derivatives of the invention are either cannabinoid (CB) receptor antagonists, CB receptor agonists, CB receptor inverse agonists or CB receptor partial agonists. The thiazole derivatives of the invention bind either on the CB₁ receptor or on the CB₂ receptor or on both the CB₁ and CB₂ receptor.

The invention relates to the use of a compound disclosed herein for the manufacture of a medicament giving a beneficial effect. A beneficial effect is disclosed herein or apparent to a person skilled in the art from the specification and general knowledge in the art. The invention also relates to the use of a compound of the invention for the manufacture of a medicament for treating or preventing a disease or condition. More particularly, the invention relates to a new use for the treatment of a disease or condition disclosed herein or apparent to a person skilled in the art from the specification and general knowledge in the art. In embodiments of the invention specific compounds disclosed herein are used for the manufacture of a medicament.

Thiazoles have been claimed in WO0127094 as triglyceride inhibitors. WO0426863 describes thiazole derivatives as transforming growth factor (TgF) inhibitors. 4,5-Diarylthiazole derivatives have been described in EP 388909 and EP 377457 as 5-lipoxygenase inhibitors for the treatment of thrombosis, hypertension, allergy and inflammation. The exemplified structures therein all contain two phenyl rings which are p-substituted with a methoxy, fluoro, methylthio or methylsulfinyl group. WO 9603392 describes sulfonylaryl-arylthiazoles for inflammation and pain, arthritis or fever as inflammation-associated disorders. JP 05345772 relates to 4,5-diarylthiazoles as acetyl cholinesterase inhibitors, and JP 04154773 describes 4,5-diarylthiazoles having analgesic, anti-inflammatory and antipyretic action.

It has now surprisingly been found that the thiazole derivatives of the formula (I), pro-drugs thereof and salts thereof 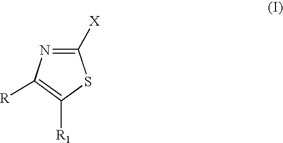
wherein
R and R₁ are the same or different and represent phenyl or pyridinyl, optionally substituted with 1-3 substituents Y, wherein Y represents a substituent from the group methyl, ethyl, propyl, methoxy, ethoxy, hydroxy, hydroxymethyl, hydroxyethyl, chloro, iodo, bromo, fluoro, trifluoromethyl, trifluoromethoxy, methylsulfonyl, methylsulfanyl, trifluoromethylsulfonyl, phenyl or cyano, with the proviso that X does not represent the subgroup (ii),
or one of the moieties R and R₁ represents a phenyl or pyridinyl group, optionally substituted with 1-3 substituents Y, wherein Y has the abovementioned meaning and the other moiety represents a hydrogen atom or a C₁₋₈ branched or linear alkyl group, C₃₋₈ branched or linear heteroalkyl group containing one heteroatom from the group (N, O, S), a C₃₋₇ cycloalkyl group, C₃₋₇-cycloalkyl-C₁₋₃-alkyl group, C₃₋₇-heterocycloalkyl-C₁₋₃-alkyl group which groups may be substituted with a hydroxy, methoxy, methyl, trifluoromethylsulfonyl or trifluoromethyl group or a fluoro atom and which C₃₋₇-heterocycloalkyl-C₁₋₃-alkyl group contains one or two heteroatoms from the group (O, N, S), or said other moiety represents a benzyl group optionally substituted on its phenyl ring with 1-3 substituents Y, wherein Y has the abovementioned meaning,
X represents one of the subgroups (i) or (ii), 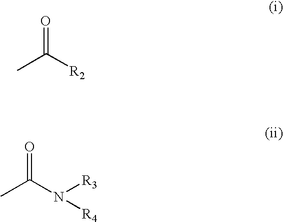
   wherein
R₂ represents a C₁₋₈ branched or linear alkyl group, C₃₋₇ cycloalkyl group, C₃₋₇-cycloalkyl-C₁₋₂-alkyl group, C₃₋₇-heterocycloalkyl-C₁₋₂-alkyl group which groups may be substituted with a hydroxy, methyl or trifluoromethyl group or a fluoro atom and which C₃₋₇-heterocycloalkyl-C₁₋₂-alkyl group contains one or two heteroatoms from the group (O, N, S), or R₂ represents a phenyl, benzyl, phenethyl or phenylpropylgroup which may be substituted on their phenyl ring with with 1-3 substituents Y, wherein Y has the abovementioned meaning, or R₂ represents a pyridyl, thienyl or naphtyl group, which napthyl group may be substituted with a halogen atom, a methyl group or a methoxy or trifluoromethyl group,,
R₃ represents a hydrogen atom or a branched or linear C₁₋₃ alkyl group,
R₄ represents hydrogen, a branched or linear C₁₋₁₀ alkyl or C₃₋₈-cycloalkyl-C₁₋₂-alkyl group, branched or linear C₁₋₁₀ alkoxy, C₃₋₈ cycloalkyl, C₅₋₁₀ bicycloalkyl, C₅₋₁₀-bicycloalkyl-C₁₋₂-alkyl, C₆₋₁₀ tricycloalkyl, C₆₋₁₀ tricycloalkyl-methyl, branched or linear C₃₋₁₀ alkenyl, C₅₋₈ cycloalkenyl, which groups may contain one or more heteroatoms from the group (O, N, S) and which groups may be substituted with a hydroxy group, 1-3 methyl groups, an ethyl group or 1-3 fluoro atoms, or R₄ represents a phenyl, phenylamino, phenoxy, benzyl, phenethyl or phenylpropyl group, optionally substituted on their phenyl ring with 1-3 substituents Y, wherein Y has the abovementioned meaning, or R₄ represents a pyridyl or thienyl group, or R₄ represents a group NR₅R₆ wherein
R₅ and R₆—together with the nitrogen atom to which they are attached—form a saturated or unsaturated, monocyclic or bicyclic, heterocyclic group having 4 to 10 ring atoms, which heterocyclic group contains one or more heteroatoms from the group (O, N, S) and which heterocyclic group may be substituted with a branched or linear C₁₋₃ alkyl, phenyl, hydroxy or trifluoromethyl group or a fluoro atom, or
R₃ and R₄—together with the nitrogen atom to which they are attached—form a saturated or unsaturated, monocyclic or bicyclic, heterocyclic group having 4 to 10 ring atoms, which heterocyclic group contains one or more heteroatoms from the group (O, N, S) and which heterocyclic group may be substituted with a branched or linear C₁₋₃ alkyl, phenyl, amino, hydroxy, methoxy, cyano or trifluoromethyl group or a fluoro or chloro atom,
   are modulators of the cannabinoid CB receptor.

To the invention belong all compounds having formula (I), racemates, mixtures of diastereomers and the individual steroisomers. Thus compounds in which the substituents on potentially asymmetrical carbon atoms are in either the R-configuration or the S-configuration belong to the invention.

Also prodrugs, i.e. compounds which when administered to humans by any known route, are metabolised to compounds having formula (I), belong to the invention. Prodrugs are bioreversible derivatives of drug molecules used to overcome some barriers to the utility of the parent drug molecule. These barriers include, but are not limited to, solubility, permeability, stability, presystemic metabolism and targeting limitations (J. Stella, “Prodrugs as therapeutics”, Expert Opin. Ther. Patents, 14(3), 277-280, 2004). In particular this relates to compounds with primary or secondary amino or hydroxy groups. Such compounds can be reacted with organic acids to yield compounds having formula (I) wherein an additional group is present which is easily removed after administration, for instance, but not limited to amidine, enamine, a Mannich base, a hydroxyl-methylene derivative, an O-(acyloxymethylene carbamate) derivative, carbamate, ester, amide or enaminone. A pro-drug is an inactive compound, which when absorbed is converted into an active form (Medicinal Chemistry: Principles and Practice, 1994, ISBN 0-85186-494-5, Ed.: F. D. King, p. 216).

Due to the CB receptor activity the compounds according to the invention are suitable for use in the treatment of psychiatric disorders such as psychosis, anxiety, depression, attention deficits, memory disorders, cognitive disorders, appetite disorders, obesity, addiction, appetence, drug dependence and neurological disorders such as neurodegenerative disorders, dementia, dystonia, muscle spasticity, tremor, epilepsy, multiple sclerosis, traumatic brain injury, stroke, Parkinson's disease, Alzheimer's disease, epilepsy, Huntington's disease, Tourette's syndrome, cerebral ischaemia, cerebral apoplexy, craniocerebral trauma, stroke, spinal cord injury, neuroinflammatory disorders, plaque sclerosis, viral encephalitis, demyelinisation related disorders, as well as for the treatment of pain disorders, including neuropathic pain disorders, and other diseases involving cannabinoid neurotransmission, including the treatment of septic shock, glaucoma, cancer, diabetes, emesis, nausea, asthma, respiratory diseases, gastrointestinal disorders, sexual disorders, gastric ulcers, diarrhoea and cardiovascular disorders.

### Pharmacological Methods

In vitro Affinity for Human Cannabinoid CB₁ Receptors

The affinity of the compounds of the invention for cannabinoid CB₁ receptors can be determined using membrane preparations of Chinese hamster ovary (CHO) cells in which the human cannabinoid CB₁ receptor is stably transfected in conjunction with [³H]CP-55,940 as radioligand. After incubation of a freshly prepared cell membrane preparation with the [³H]-ligand, with or without addition of compounds of the invention, separation of bound and free ligand is performed by filtration over glassfiber filters. Radioactivity on the filter is measured by liquid scintillation counting.

In Vitro Affinity for Human Cannabinoid CB₂ Receptors

The affinity of the compounds of the invention for cannabinoid CB₂ receptors can be determined using membrane preparations of Chinese hamster ovary (CHO) cells in which the human cannabinoid CB₂ receptor is stably transfected in conjunction with [³H]CP-55,940 as radioligand. After incubation of a freshly prepared cell membrane preparation with the [³H]-ligand, with or without addition of compounds of the invention, separation of bound and free ligand is performed by filtration over glassfiber filters. Radioactivity on the filter is measured by liquid scintillation counting.

In Vitro Antagonism on Human Cannabinoid CB₁ Receptors

In vitro CB₁ receptor antagonism can be assessed with the human CB₁ receptor cloned in Chinese hamster ovary (CHO) cells. CHO cells are grown in a Dulbecco's Modified Eagle's medium (DMEM) culture medium, supplemented with 10% heat-inactivated fetal calf serum. Medium is aspirated and replaced by DMEM, without fetal calf serum, but containing [³H]-arachidonic acid and incubated overnight in a cell culture stove (5% CO₂/95% air; 37° C.; water-saturated atmosphere). During this period [³H]-arachidonic acid is incorporated in membrane phospholipids. On the test day, medium is aspirated and cells are washed three times using 0.5 mL DMEM, containing 0.2% bovine serum albumin (BSA). Stimulation of the CB₁ receptor by WIN 55,212-2 leads to activation of PLA₂ followed by release of [³H]-arachidonic acid into the medium. This WIN 55,212-2-induced release is concentration-dependently antagonized by CB₁ receptor antagonists. The CB₁ antagonistic potencies of the test compounds are expressed as pA₂ values.

In Vivo Antagonism on Human Cannabinoid CB, Receptors

In vivo CB₁ antagonism can be assessed with the CP-55,940-induced hypotension test in rat. Male normotensive rats (225-300 g; Harlan, Horst, The Netherlands) are anaesthetized with pentobarbital (80 mg/kg ip). Blood pressure is measured, via a cannula inserted into the left carotid artery, by means of a Spectramed DTX-plus pressure transducer (Spectramed B.V., Bilthoven, The Netherlands). After amplification by a Nihon Kohden Carrier Amplifier (Type AP-621G; Nihon Kohden B.V., Amsterdam, The Netherlands), the blood pressure signal is registered on a personal computer (Compaq Deskpro 386s), by means of a Po-Ne-Mah data-acquisition program (Po-Ne-Mah Inc., Storrs, USA). Heart rate is derived from the pulsatile pressure signal. All compounds are administered orally as a microsuspension in 1% methylcellulose 30 minutes before induction of the anesthesia which is 60 minutes prior to administration of the CB₁ receptor agonist CP-55,940. The injection volume is 10 mL kg⁻¹. After haemodynamic stabilization the CB₁ receptor agonist CP-55,940 (0.1 mg kg⁻¹ i.v.) is administered and the hypotensive effect established. (Wagner, J. A. et al., Hemodynamic effects of cannabinoids: coronary and cerebral vasodilation mediated by cannabinoid CB1 receptors. Eur. J. Pharmacol. 2001, 423, 203-210).

This hypotension test can also be used to assess CB₁ receptor agonistic effects of the compounds. Such CB₁ agonistic effects on blood pressure may be counteracted by a selective CB₁ receptor antagonist such as rimonabant.

Cannabinoid receptor agonistic or partial agonistic activity of compounds of the invention can be determined according to published methods, such as assessment of in vivo cannabimimetic effects (Wiley, J. L. et al., J. Pharmacol. Exp. Ther. 2001, 296, 1013).

The compounds of the invention can be brought into forms suitable for administration by means of usual processes using auxiliary substances and/or liquid or solid carrier materials.

Compounds of the present invention are generally administered as pharmaceutical compositions which are important and novel embodiments of the invention because of the presence of the compounds, more particularly specific compounds disclosed herein. Types of pharmaceutical compositions that may be used include but are not limited to tablets, chewable tablets, capsules, solutions, parenteral solutions, suppositories, suspensions, and other types disclosed herein or apparent to a person skilled in the art from the specification and general knowledge in the art.

In embodiments of the invention, a pharmaceutical pack or kit is provided comprising one or more containers filled with one or more of the ingredients of a pharmaceutical composition of the invention. Associated with such container(s) can be various written materials such as instructions for use, or a notice in the form prescribed by a governmental agency regulating the manufacture, use or sale of pharmaceuticals products, which notice reflects approval by the agency of manufacture, use, or sale for human or veterinary administration.

General Aspects of Syntheses

Thiazole derivatives can be obtained according to methods known, for example.
a) Organic Reactions, Vol. VI, (1951), p. 367-409, Ed. R. Adams, John Wiley and Sons Inc., New York
b) J. S. Carter et al., Bioorg. Med. Chem. Lett. (1999), 9, 1167-1170
c) T. T. Sakai et al., Bioorg. Med. Chem. (1999), 7, 1559-1566
d) A. Tanaka et al., J. Med. Chem. (1994), 37, 1189-1199
e) J. J. Talley et al., WO 9603392: Chem. Abstr. 125, 33628
f) V. Cecchetti et al., Bioorg. Med. Chem. (1994), 2, 799-806
g) T. Eicher et al., The Chemistry of Heterocycles, (1995) p. 149-155, Georg Thieme Verlag, Stuttgart, 1995, ISBN 313-100511-4, and references cited therein
h) Gilchrist, T. L., Heterocyclic Chemistry, 3th Ed. 1997, p. 319-327, Longman, UK, ISBN 0-582-27843-0.

Alfahaloketones can be obtained by halogenation of the corresponding ketone. The reaction of alfa-halo carbonyl compounds and thioamide can produce a wide range of thiazole derivatives. More in particular, condensation of alfa-bromoketones with ethyl thiooxamate provides (2-ethoxy-carbonyl)thiazoles of general formula (II).

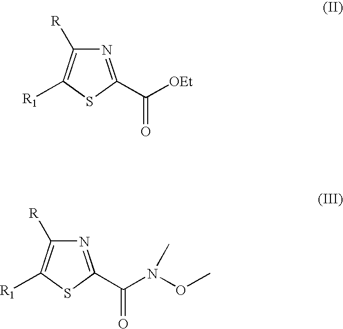

Compounds of formula (II) can be converted to the corresponding N-methoxy-N-methylamide (III) and subsequently reacted with an alkyllithium or aryl lithium reagent to give a compound of general formula (I), wherein X represents subgroup (i).

Compounds of general formula (II) can be amidated with an amine of general formula R₃R₄NH into a compound of general formula (I) wherein X represents subgroup (ii). Such amidations can be catalyzed by (CH₃)₃Al. (For more information on aluminum-mediated conversion of esters to amides, see: J. I. Levin, E. Turos, S. M. Weinreb, Synth. Commun. (1982), 12, 989-993.)

Alternatively, a compound having formula (II) is converted into the corresponding carboxylic acid and subsequently reacted with a so-called halogenating agent such as for example thionyl chloride (SOCl₂). This reaction gives the corresponding carbonyl chloride that is subsequently reacted with a compound having formula R₃R₄NH wherein R₃ and R₄ have the meanings as described above.

Alternatively, the ester group in (II) can be converted to the corresponding carboxylic acid. This carboxylic acid can be reacted with a compound having formula R₃R₄NH wherein R₃ and R₄ have the meanings as described hereinabove via activating and coupling methods such as formation of an active ester, or in the presence of a so-called coupling reagent, such as for example, DCC, HBTU, HOAT (N-hydroxy-7-azabenzotriazole), BOP, CIP (2-chloro-1,3-dimethylimidazolinium hexafluorophosphate), PyAOP (7-azabenzotriazol-1-yloxytris(pyrrolidino)-phosphonium hexafluorophosphate) and the like. (For more information on activating and coupling methods see a) M. Bodanszky, A. Bodanszky: The Practice of Peptide Synthesis, Springer-Verlag, New York, 1994; ISBN: 0-387-57505-7; b) K. Akaji et al., Tetrahedron Lett. (1994), 35, 3315-3318; c) F. Albericio et al., Tetrahedron Lett. (1997), 38, 4853-4856).

According to these procedures the following compounds can be prepared. They are intended to further illustrate the invention in more detail, and therefore are not deemed to restrict the scope of the invention in any way.

### SYNTHESES OF SPECIFIC EXAMPLES

¹H NMR spectra were recorded on a Varian UN400 instrument (400 MHz) with tetramethylsilane as an internal standard. Chemical shifts are given in ppm (δ scale) downfield from tetramethylsilane. Coupling constants (J) are expressed in Hz. Thin-layer chromatography was performed on Merck pre-coated 60 F₂₅₄ plates, and spots were visualised with UV light. Flash chromatography was performed using silica gel 60 (0.040-0.063 mm, Merck). Column chromatography was performed using silica gel 60 (0.063-0.200 mm, Merck). Melting points were recorded on a Büchi B-545 melting point apparatus and are uncorrected.

### Example 1

Part A: To a solution of 1-(2,4-dichlorophenyl)-2-phenylethanone (54.35 gram, 0.205 mol) in benzene (220 mL) is slowly added bromine (10.6 mL, 0.205 mol) and the resulting solution is stirred at room temperature for 1 hour. Aqueous (5%) NaHCO₃ solution is slowly added. The organic layer is separated, dried over MgSO₄, filtered and evaporated in vacuo to give crude 2-bromo-1-(2,4-dichlorophenyl)-2-phenylethanone (69.4 g, 98% yield) as an oil. ¹H-NMR (400 MHz, CDCl₃): δ 6.20 (s, 1H), 7.26 (dd, J=8 and 2 Hz, 1H), 7.31-7.50 (m, 7H).

Part B; 2-Bromo-1-(2,4-dichlorophenyl)-2-phenylethanone (25.83 gram, 0.075 mol) and ethyl thiooxamate (15.0 gram, 0.112 mol) are dissolved in absolute ethanol (200 mL). The resulting mixture is heated at reflux temperature for 16 hours. After evaporation in vacuo the crude material is dissolved in a mixture of water and dichloromethane. The dichloromethane layer is separated and the water layer is extracted three times with dichloromethane. The collected organic layers are dried (MgSO₄), filtered and concentrated. The resulting material is purified by column chromatography (silica gel/dichloromethane) to give ethyl 4-(2,4-dichlorophenyl)-5-phenylthiazole-2-carboxylate (10.5 gram, 37% yield). ¹H-NMR (400 MHz, CDCl₃): δ 1.46 (t, J=7 Hz, 3H), 4.53 (q, J=7 Hz, 2H), 7.24-7.38 (m, 7H), 7.43 (d, J=2 Hz, 1H).

Part C: To a solution of ethyl 4-(2,4-dichlorophenyl)-5-phenylthiazole-2-carboxylate (10.5 g, 0.028 mol) in methanol (170 ml) is slowly added a solution of KOH (8.9 g, 0.0896 mol) in water (170 ml). The resulting solution is heated at 90° C. for 2 hours and cooled to room temperature. A mixture of concentrated HCl and ice is added. The formed precipitate is collected, washed with water and diethyl ether and dried to give 4-(2,4-dichlorophenyl)-5-phenylthiazole-2-carboxylic acid (8.99 gram, 92% yield). Melting point: 105° C.

Part D: To a magnetically stirred suspension of 4-(2,4-dichlorophenyl)-5-phenylthiazole-2-carboxylic acid (4.2 g, 0.012 mol) in anhydrous dichloromethane (170 ml) is successively added 7-aza-1-hydroxybenzotriazole (HOAT). (4.083 gram, 0.030 mol), 7-azabenzotriazol-1-yloxytris(pyrrolidino)phosphonium hexafluorophos-phate (PyAOP) (15.64 gram, 0.03 mol), diisopropylethylamine (6.26 ml, 0.036 mol) and N-methoxy-N-methylamine HCl (2.925 gram, 0.030 mol) and the resulting solution is stirred for 16 hours at room temperature. A 5% aqueous NaHCO₃ solution is slowly added and the resulting mixture is extracted (3×) with dichloromethane. The collected organic layers are dried (MgSO₄), filtered and concentrated to give a crude oil (18.9 gram). Purification by flash chromatography (silica gel/ethyl acetate/petroleum ether=1/1) gives N-methyl-N-methoxy-4-(2,4-dichlorophenyl)-5-phenylthiazole-2-carboxamide (4.0 gram, 85% yield). ¹H-NMR (400 MHz, CDCl₃): δ 3.60 (br s, 3H), 3.90 (s, 3H), 7.21-7.33 (m, 7H), 7.45 (d, J=2 Hz, 1H).

Analogously was prepared: N-methyl-N-methoxy-4-(2-chlorophenyl)-5-phenylthiazole-2-carboxamide. ¹H-NMR (400 MHz, CDCl₃): δ 3.62 (br s, 3H), 3.90 (s, 3H), 7.22-7.45 (m, 9H).

Part E: To a cooled (−70° C.) and stirred solution of N-methyl-N-methoxy4-(2,4-dichlorophenyl)-5-phenylthiazole-2-carboxamide (2.0 gram, 0.005 mol) in THF (20 ml) under N₂ is added n-BuLi (3.13 ml, 1.6 M solution in hexane, 0.005 mol). After stirring for 30 minutes the solution is allowed to attain room temperature and stirred for 16 hours. Aqueous HCl (20 ml, 1N) is added and the resulting mixture is extracted with diethyl ether. The diethyl ether layers are washed with water (2×), dried (MgSO₄), filtered and concentrated to give a crude oil (2.03 gram). Purification by flash chromatography (silica gel/dichloromethane) gives 1-[4-(2,4-dichlorophenyl)-5-phenylthiazol-2-yl]pentan-1-one (0.6 gram, 31% yield). ¹H-NMR (400 MHz, CDCl₃): δ 0.95 (t, J=7 Hz, 3H), 1.38-1.48 (m, 2H), 1.72-1.80 (m, 2H), 3.16 (t, J˜7 Hz, 2H), 7.20-7.35 (m, 7H), 7.46 (d, J=2Hz, 1H).

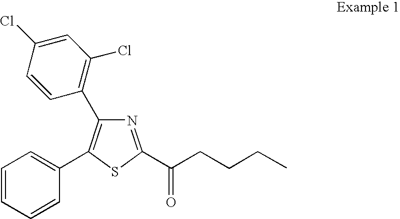

Analogously were prepared:

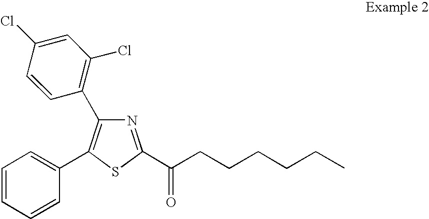

### Example 2

### ¹H-NMR (400 MHz, CDCl₃): δ 0.93 (t, J=7 Hz, 3H), 1.26-1.44 (m, 6H), 1.73-1.82 (m, 2H), 3.15 (t, J˜7 Hz, 2H), 7.21-7.34 (m, 7H), 7.47 (d, J=2 Hz, 1H)

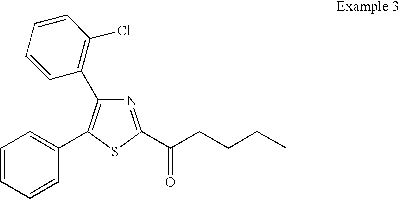

### Example 3

### Melting Point: 78-80° C.

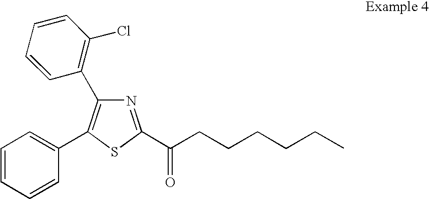

### Example 4

### ¹H-NMR (400 MHz, CDCl₃): δ 0.93 (t, J=7 Hz, 3H), 1.25-1.44 (m, 6H), 1.73-1.82 (m, 2H), 3.17 (t, J˜7 Hz, 2H), 7.22-7.40 (m, 8H), 7.47 (dd, J=8 and 2 Hz, 1H)

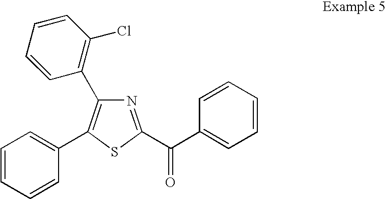

### Example 5

### Melting Point: 131-132° C.

### Example 6

Part A: To a magnetically stirred solution of 1-phenylheptan-1-one (23.7 gram, 0.125 mol) in benzene (160 mL) is slowly added bromine (7.0 mL, 0.125 mol) and the resulting solution is reacted at room temperature for 1 hour. Aqueous (5%) NaHCO₃ solution is slowly added, followed by dichloromethane. The organic layer is separated, dried over MgSO₄, filtered and evaporated in vacuo to give crude 2-bromo-1-phenylheptan-1-one (41.8 g, quantitative yield) as an oil. ¹H-NMR (400 MHz, CDCl₃): δ 0.90 (t, J=7 Hz, 3H), 1.28-1.78 (m, 6H), 2.04-2.25 (m, 2H), 5.11-5.16 (m, 1H), 7.42-7.62 (m, 3H), 8.00-8.04 (m, 2H).

Part B; 2-Bromo-1-phenylheptan-1-one (20.17 gram, 0.075 mol) and ethyl thiooxamate (15.0 gram, 0.112 mol) are dissolved in absolute ethanol (200 mL). The resulting mixture is heated at reflux temperature for 16 hours. After evaporation in vacuo the crude material is dissolved in a mixture of water and dichloromethane. The dichloromethane layer is separated and the water layer is extracted three times with dichloromethane. The collected organic layers are dried (MgSO₄), filtered and concentrated. The resulting material is purified by column chromatography (silica gel/dichloromethane/petroleum ether=1/1) to give ethyl 5-(n-pentyl)-4-phenylthiazole-2-carboxylate (12.09 gram, 53% yield) as an oil which slowly solidified. Melting point: 51-52° C.

Part C: To a magnetically stirred solution of ethyl 5-(n-pentyl)-4-phenylthiazole-2-carboxylate (12.09 g, 0.039 mol) in methanol (240 ml) is slowly added a solution of KOH (8.9 g) in water (240 ml). The resulting solution is heated at reflux temperature for 2 hours and subsequently cooled to room temperature. A mixture of concentrated HCl and ice is added. The formed precipitate is collected, successively washed with water and cold diethyl ether and dried to give 5-(n-pentyl)-4-phenylthiazole-2-carboxylic acid (3.54 gram, 32% yield). ¹H-NMR (400 MHz, CDCl₃): δ 0.87 (t, J=7 Hz, 3H), 1.28-1.40 (m, 4H), 1.66-1.76 (m, 2H), 2.93-3.00 (m, 2H), 4.00 (br s, 1H), 7.35-7.46 (m, 3H), 7.58-7.64 (m, 2H).

Part D: To a magnetically stirred suspension of 5-(n-pentyl)-4-phenylthiazole-2-carboxylic acid (1.18 g, 0.0043 mol) in anhydrous dichloromethane (35 ml) is successively added 7-aza-1-hydroxybenzotriazole (HOAT) (1.46 gram, 0.0107 mol), 7-azabenzotriazol-1-yloxytris(pyrrolidino)phosphonium hexafluorophosphate (PyAOP) (5.59 gram, 0.0107 mol), diisopropylethylamine (2.24 ml, 0.0129 mol) and aniline (0.98 ml, 0.0107 mol) and the resulting solution is stirred for 16 hours at room temperature. The resulting mixture is concentrated and purified by flash chromatography (silica gel/dichloromethane) to give N-phenyl-5-(n-pentyl)-4-phenylthiazole-2-carboxamide (0.89 gram, 59% yield). ¹H-NMR (400 MHz, CDCl₃): δ 0.98 (t, J=7 Hz, 3H), 1.26-1.41 (m, 4H), 1.68-1.78 (m, 2H), 2.97 (t, J=7 Hz, 2H), 7.12-7.18 (m, 1H), 7.34-7.52 (m, 5H), 7.60-7.64 (m, 2H), 7.69-7.74 (m, 2H), 9.10 (br s 1H).

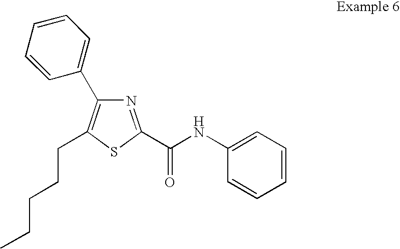

Analogously were prepared: 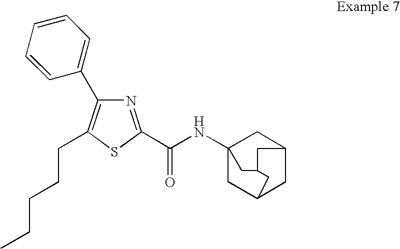
from 5-(n-pentyl)-4-phenylthiazole-2-carboxylic acid and 1-aminoadamantane.

### Example 7

### Melting Point: 90-92° C.

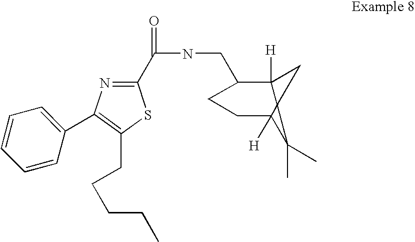 from 5-(n-pentyl)-4-phenylthiazole-2-carboxylic acid and cis-myrtanylamine (CAS 38235-68-6)

### Example 8

### ¹H-NMR (400 MHz, CDCl₃): δ 0.89 (t, J=7 Hz, 3H), 1.08 (s, 3H), 1.20 (s, 3H), 1.26-1.38 (m, 4H), 1.50-1.62 (m, 1H), 1.66-1.74 (m, 2H), 1.82-2.04 (m, 5H), 2.28-2.40 (m, 2H), 2.94 (t, J=7 Hz, 2H), 3.39-3.50 (m, 2H), 7.29 (br t, J˜7 Hz, 1 H), 7.37-7.60 (m, 5H)

### Example 9

Part A: 1-Bromo-1-phenylheptan-2-one (19.98 gram, 0.074 mol) and ethyl thiooxamate (15.0 gram, 0.112 mol) are dissolved in absolute ethanol (200 mL). The resulting mixture is heated at reflux temperature for 2 hours. After evaporation in vacuo the crude material is dissolved in a mixture of water and dichloromethane. The dichloromethane layer is separated and the water layer is extracted three times with dichloromethane. The collected organic layers are dried (MgSO₄), filtered and concentrated. The resulting material is purified by column chromatography (silica gel/dichloromethane/petroleum ether=1/1) to give ethyl 4-(n-pentyl)-5-phenylthiazole-2-carboxylate (5.24 gram, 23% yield) as an oil. ¹H-NMR (400 MHz, CDCl₃): δ 0.89 (t, J=7 Hz, 3H), 1.24-1.32 (m, 4H), 1.44 (t, J=7 Hz, 3H), 1.70-1.78 (m, 2H), 2.81-2.87 (m, 2H), 4.48 (q, J=7 Hz, 2H), 7.40-7.48 (m, 5H).

Analogously were prepared:

Ethyl 4-benzyl-5-phenylthiazole-2-carboxylate as an oil.

Ethyl 5-(n-pentyl)-4-(2,4-dichorophenyl)thiazole-2-carboxylate. Melting point: 92-93° C.

Part B: To a magnetically stirred solution of 1-aminoadamantane (1.607 gram, 0.0086 mol) in anhydrous dichloromethane (10 ml) is added Al(CH₃)₃ (4.3 mL, 2M solution in hexane, 0.0086 mol) and the resulting solution is reacted at room temperature for 10 minutes. Aqueous (5%) NaHCO₃ solution is slowly added. Extraction with dichloromethane, drying over MgSO₄, filtration and concentration in vacuo, followed by column chromatography (silica gel/dichloromethane) gives N-(adamant-1-yl)-4-(n-pentyl)-5-phenylthiazole-2-carboxamide (1.17 g, 72% yield). ¹H-NMR (400 MHz, CDCl₃): δ 0.88 (t, J=7 Hz, 3H), 1.26-1.34 (m, 4H), 1.68-1.78 (m, 8H), 2.08-2.18 (m, 9H), 2.71-2.7 6 (m, 2H), 7.02 (br s, 1H), 7.36-7.45 (m, 5H).

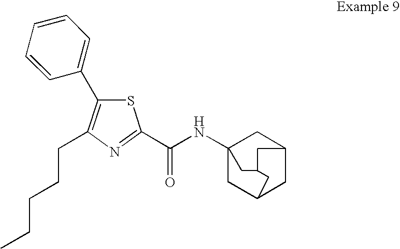

Analogously were prepared: 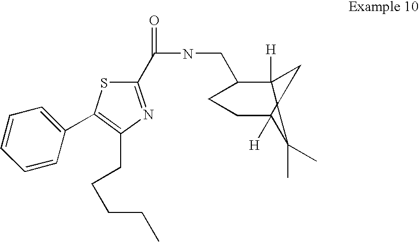
from ethyl 4-(n-pentyl)-5-phenylthiazole-2-carboxylate and cis-myrtanylamine (CAS 38235-68-6)

### Example 10

### ¹H-NMR (400 MHz, CDCl₃): δ 0.89 (t, J=7 Hz, 3H), 1.10 (s, 3H), 1.22 (s, 3H), 1.25-1.34 (m, 4H), 1.54-1.78 (m, 3H), 1.84-2.06 (m, 5H), 2.31-2.42 (m, 2H), 2.72-2.78 (m, 2H), 3.44-3.50 (m, 2H), 7.24-7.28 (m, 1H), 7.37-7.46 (m, 5H)

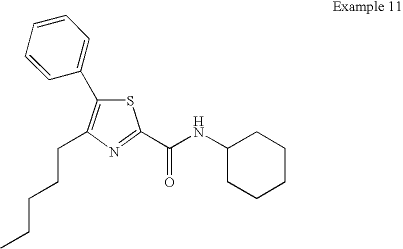 from ethyl 4-(n-pentyl)-5-phenylthiazole-2-carboxylate and cyclohexylamine

### Example 11

### Melting Point: 84-85° C.

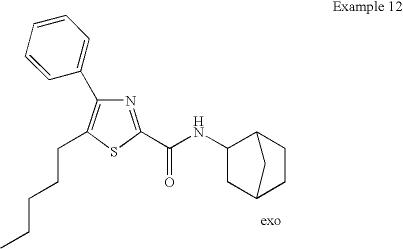 from ethyl 5-(n-pentyl)-4-phenylthiazole-2-carboxylate and exo-2-amino-bicyclo[2.2.1]heptane

### Example 12

### Melting Point: 64-65° C.

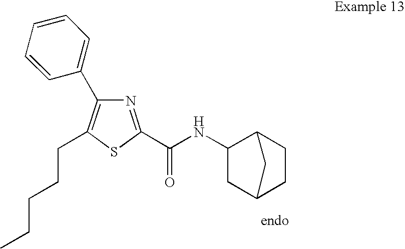 from ethyl 5-(n-pentyl)-4-phenylthiazole-2-carboxylate and endo-2-amino-bicyclo[2.2.1]heptane

### Example 13

### Melting Point: 80-82° C.

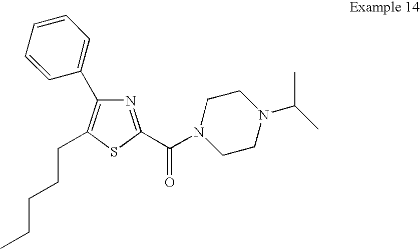 from ethyl 5-(n-pentyl)-4-phenylthiazole-2-carboxylate and 4-isopropylpiperazine

### Example 14

### Melting Point: 84-85° C.

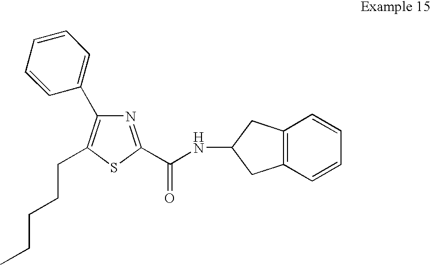 from ethyl 5-(n-pentyl)-4-phenylthiazole-2-carboxylate and indan-2-ylamine

### Example 15

### ¹H-NMR (400 MHz, CDCl₃): δ 0.85 (t, J=7 Hz, 3H), 1.26-1.38 (m, 4H), 1.65-1.75 (m, 2H), 2.90-3.01 (m, 4H), 3.36-3.44 (m, 2H), 4.86-4.96 (m, 1H), 7.15-7.27 (m, 4H), 7.35-7.47 (m, 4H), 7.52-7.56 (m, 2H)

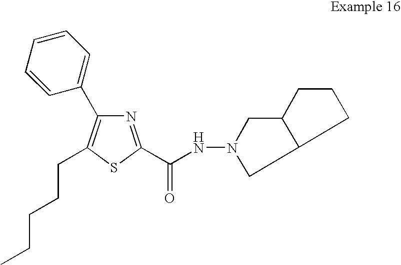 from ethyl 5-(n-pentyl)-4-phenylthiazole-2-carboxylate and 3-aza-bicyclo[3.3.0]octane

### Example 16

### Melting Point: 86-87° C.

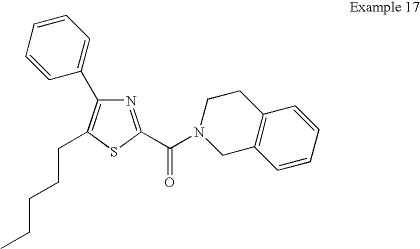 from ethyl 5-(n-pentyl)-4-phenylthiazole-2-carboxylate and 1,2,3,4-tetrahydroisoquinoline

### Example 17

### Melting Point: 50-51° C.

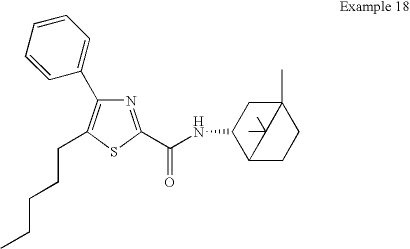 from ethyl 5-(n-pentyl)-4-phenylthiazole-2-carboxylate and R-(+)-bornylamine (CAS 32511-34-5).

### Example 18

### ¹H-NMR (400 MHz, CDCl₃): δ 0.90-1.02 (m, 13H), 1.22-1.47 (m, 6H), 1.60-1.84 (m, 5H), 2.36-2.45 (m, 1H), 2.94 (t, J=7 Hz, 2H), 4.36-4.44 (m, 1H), 7.32 (br d, J˜7 Hz, 1H), 7.38-7.50 (m, 3H), 7.58-7.63 (m, 2H)

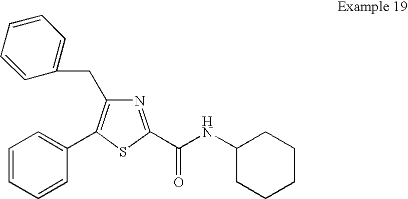 from ethyl 4-benzyl-5-phenylthiazole-2-carboxylate and cyclohexylamine

### Example 19

### Melting Point: 104-106° C.

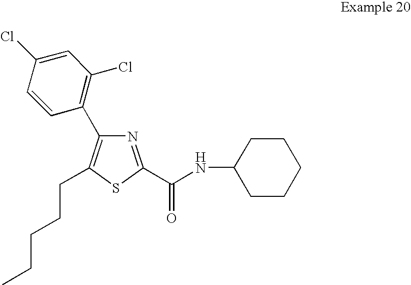 from ethyl 5-(n-pentyl)-4-(2,4-dichlorophenyl)thiazole-2-carboxylate and cyclohexylamine

### Example 20

### ¹H-NMR (400 MHz, CDCl₃): δ 0.85 (t, J=7 Hz, 3H), 1.13-1.46 (m, 8H), 1.55-1.68 (m, 4H), 1.71-1.80 (m, 2H), 1.96-2.06 (m, 2H), 2.67 (t, J=7 Hz, 2H), 3.86-3.98 (m, 1H), 7.05 (br d, J=7 Hz, 1H), 7.26-7.37 (m, 2H), 7.53 (d, J=2 Hz, 1H)

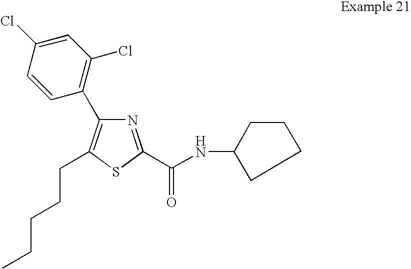 from ethyl 5-(n-pentyl)-4-(2,4-dichlorophenyl)thiazole-2-carboxylate and cyclopentylamine

### Example 21

### ¹H-NMR (400 MHz, CDCl₃): δ 0.85 (t, J=7 Hz, 3H), 1.20-1.30 (m, 4H), 1.48-1.77 (m, 8H), 2.01-2.10 (m, 2H), 2.67 (t, J=7 Hz, 2H), 4.31-4.41 (m, 1H), 7.09 (br d, J=7 Hz, 1H), 7.25-7.37 (m, 2H), 7.53 (d, J=2 Hz, 1H)

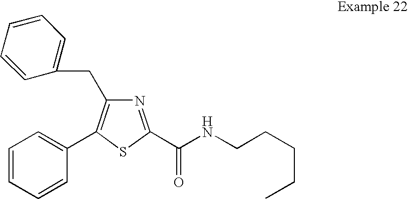 from ethyl 4-benzyl-5-phenylthiazole-2-carboxylate and n-pentylamine

### Example 22

### ¹H-NMR (400 MHz, CDCl₃): δ 0.89 (t, J=7 Hz, 3H), 1.33-1.40 (m, 4H), 1.59-1.67 (m, 2H), 3.40-3.47 (m, 2H), 4.16 (s, 2H), 7.15-7.32 (m, 6H), 7.39-7.42 (m, 5H)

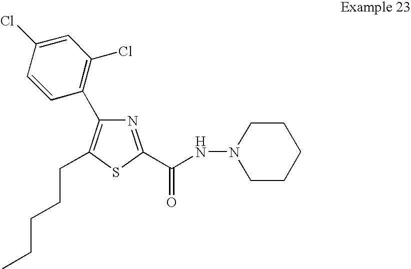 from ethyl 5-(n-pentyl)-4-(2,4-dichlorophenyl)thiazole-2-carboxylate and 1-aminopiperidine

### Example 23

### ¹H-NMR (400 MHz, CDCl₃): δ 0.85 (t, J=7 Hz, 3H), 1.20-1.28 (m, 4H), 1.39-1.46 (m, 2H), 1.56-1.64 (m, 2H), 1.71-1.79 (m, 4H), 2.66 (t, J=7 Hz, 2H), 2.82-2.88 (m, 4H), 7.29 (d, J=8 Hz, 1H), 7.35 (dd, J=8 and 2 Hz, 1H), 7.53 (d, J=2 Hz, 1H), 7.88 (br s, 1H)

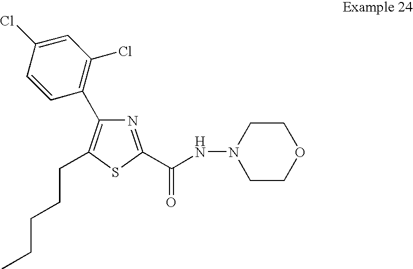 from ethyl 5-(n-pentyl)-4-(2,4-dichlorophenyl)thiazole-2-carboxylate and 4-aminomorpholine

### Example 24

### ¹H-NMR (400 MHz, CDCl₃): δ 0.85 (t, J=7 Hz, 3H), 1.21-1.29 (m, 4H), 1.57-1.66 (m, 2H), 2.67 (t, J=7 Hz, 2H), 2.93-2.98 (m, 4H), 3.82-3.88 (m, 4H), 7.29 (d, J=8 Hz, 1H), 7.35 (dd, J=8 and 2 Hz, 1H), 7.54 (d, J=2 Hz, 1H), 7.95 (br s, 1H)

Pharmacological TestResults

Receptor affinity

| | Human CB₁ | Human CB₂ |
|---|---|---|
| Compound | pKᵢ-value | pKᵢ-value |
| Example 1 | 6.6 | |
| Example 4 | 6.8 | |
| Example 7 | 7.3 | |
| Example 8 | 6.8 | |
| Example 9 | 7.8 | 8.1 |
| Example 10 | 6.9 | 8.1 |
| Example 11 | 6.6 | 7.7 |

1. Use of a compound of formula (I) 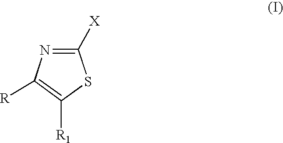
wherein
R and R₁ are the same or different and represent phenyl or pyridinyl, optionally substituted with 1-3 substituents Y, wherein Y represents a substituent from the group methyl, ethyl, propyl, methoxy, ethoxy, hydroxy, hydroxymethyl, hydroxyethyl, chloro, iodo, bromo, fluoro, trifluoromethyl, trifluoromethoxy, methylsulfonyl, methylsulfanyl, trifluoromethylsulfonyl, phenyl or cyano, with the proviso that X does not represent the subgroup (ii),
or one of the moieties R and R₁ represents a phenyl or pyridinyl group, optionally substituted with 1-3 substituents Y, wherein Y has the abovementioned meaning and the other moiety represents a hydrogen atom or a C₁₋₈ branched or linear alkyl group, C₃₋₈ branched or linear heteroalkyl group containing one heteroatom from the group (N, O, S), a C₃₋₇ cycloalkyl group, C₃₋₇-cycloalkyl-C₁₋₃-alkyl group, C₃₋₇-heterocycloalkyl-C₁₋₃-alkyl group which groups may be substituted with a hydroxy, methoxy, methyl, trifluoromethylsulfonyl or trifluoromethyl group or a fluoro atom and which C₃₋₇-heterocycloalkyl-C₁₋₃-alkyl group contains one or two heteroatoms from the group (O, N, S), or said other moiety represents a benzyl group optionally substituted on its phenyl ring with 1-3 substituents Y, wherein Y has the abovementioned meaning,
X represents one of the subgroups (i) or (ii), 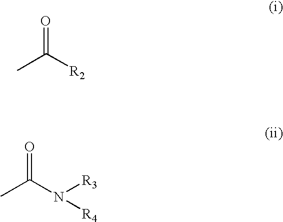
wherein
R₂ represents a C₁₋₈ branched or linear alkyl group, C₃₋₇ cycloalkyl group, C₃₋₇-cycloalkyl-C₁₋₂-alkyl group, C₃₋₇-heterocycloalkyl-C₁₋₂-alkyl group which groups may be substituted with a hydroxy, methyl or trifluoromethyl group or a fluoro atom and which C₃₋₇-heterocycloalkyl-C₁₋₂-alkyl group contains one or two heteroatoms from the group (O, N, S), or R₂ represents a phenyl, benzyl, phenethyl or phenylpropylgroup which may be substituted on their phenyl ring with with 1-3 substituents Y, wherein Y has the abovementioned meaning, or R₂ represents a pyridyl, thienyl or naphtyl group, which napthyl group may be substituted with a halogen atom, a methyl group or a methoxy or trifluoromethyl group,,
R₃ represents a hydrogen atom or a branched or linear C₁₋₃ alkyl group,
R₄ represents hydrogen, a branched or linear C₁₋₁₀ alkyl or C₃₋₈-cycloalkyl-C₁₋₂-alkyl group, branched or linear C₁₋₁₀ alkoxy, C₃₋₈ cycloalkyl, C₅₋₁₀ bicycloalkyl, C₅₋₁₀-bicycloalkyl-C₁₋₂-alkyl, C₆₋₁₀ tricycloalkyl, C₆₋₁₀ tricycloalkyl-methyl, branched or linear C₃₋₁₀ alkenyl, C₅₋₈ cycloalkenyl, which groups may contain one or more heteroatoms from the group (O, N, S) and which groups may be substituted with a hydroxy group, 1-3 methyl groups, an ethyl group or 1-3 fluoro atoms, or R₄ represents a phenyl, phenylamino, phenoxy, benzyl, phenethyl or phenylpropyl group, optionally substituted on their phenyl ring with 1-3 substituents Y, wherein Y has the abovementioned meaning, or R₄ represents a pyridyl or thienyl group, or R₄ represents a group NR₅R₆ wherein
R₅ and R₆—together with the nitrogen atom to which they are attached—form a saturated or unsaturated, monocyclic or bicyclic, heterocyclic group having 4 to 10 ring atoms, which heterocyclic group contains one or more heteroatoms from the group (O, N, S) and which heterocyclic group may be substituted with a branched or linear C₁₋₃ alkyl, phenyl, hydroxy or trifluoromethyl group or a fluoro atom, or
R₃ and R₄—together with the nitrogen atom to which they are attached—form a saturated or unsaturated, monocyclic or bicyclic, heterocyclic group having 4 to 10 ring atoms, which heterocyclic group contains one or more heteroatoms from the group (O, N, S) and which heterocyclic group may be substituted with a branched or linear C₁₋₃ alkyl, phenyl, amino, hydroxy, methoxy, cyano or trifluoromethyl group or a fluoro or chloro atom,
and pharmacologically acceptable salts thereof, as well as prodrugs, for the preparation of a pharmaceutical composition for the treatment of disorders involving cannabinoid neurotransmission such as psychosis, anxiety, depression, attention deficits, memory disorders, cognitive disorders, appetite disorders, obesity, addiction, appetence, drug dependence and neurological disorders such as neurodegenerative disorders, dementia, dystonia, muscle spasticity, tremor, epilepsy, multiple sclerosis, traumatic brain injury, stroke, Parkinson's disease, Alzheimer's disease, epilepsy, Huntington's disease, Tourette's syndrome, cerebral ischaemia, cerebral apoplexy, craniocerebral trauma, stroke, spinal cord injury, neuroinflammatory disorders, plaque sclerosis, viral encephalitis, demyelinisation related disorders, as well as for the treatment of pain disorders, including neuropathic pain disorders, and other diseases involving cannabinoid neurotransmission, including the treatment of septic shock, glaucoma, cancer, diabetes, emesis, nausea, asthma, respiratory diseases, gastrointestinal disorders, sexual disorders, gastric ulcers, diarrhoea and cardiovascular disorders.

2. A compound of formula (I) 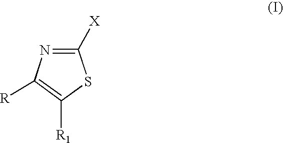
wherein
R and R₁ are the same or different and represent phenyl, 3-pyridinyl or 4-pyridinyl, optionally substituted with 1-3 substituents Y, wherein Y represents a substituent from the group methyl, ethyl, propyl, methoxy, ethoxy, hydroxy, hydroxymethyl, hydroxyethyl, chloro, iodo, bromo, fluoro, trifluoromethyl, trifluoromethoxy, methylsulfonyl, methylsulfanyl, trifluoromethylsulfonyl, phenyl or cyano, with the proviso that X does not represent the subgroup (ii),
or one of the moieties R and R₁ represents a phenyl, 3-pyridinyl or 4-pyridinyl group, optionally substituted with 1-3 substituents Y, wherein Y has the abovementioned meaning and the other moiety represents a C₂₋₈ branched or linear alkyl group, C₃₋₈ branched or linear heteroalkyl group containing one heteroatom from the group (N, O, S), a C₃₋₇ cycloalkyl group, C₃₋₇-cycloalkyl-C₁₋₃-alkyl group, C₃₋₇-heterocycloalkyl-C₁₋₃-alkyl group which groups may be substituted with a hydroxy, methoxy, methyl, trifluoromethylsulfonyl or trifluoromethyl group or a fluoro atom and which C₃₋₇-heterocycloalkyl-C₁₋₃-alkyl group contains one or two heteroatoms from the group (O, N, S), or said other moiety represents a benzyl group optionally substituted on its phenyl ring with 1-3 substituents Y, wherein Y has the abovementioned meaning,
X represents one of the subgroups (i) or (ii), 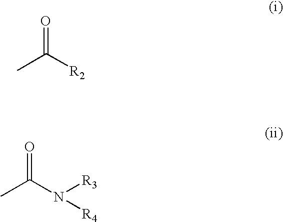
wherein
R₂ represents a C₃₋₈ branched or linear alkyl group, C₃₋₇ cycloalkyl group, C₃₋₇-cycloalkyl-C₁₋₂-alkyl group, C₃₋₇-heterocycloalkyl-C₁₋₂-alkyl group which groups may be substituted with a hydroxy, methyl or trifluoromethyl group or a fluoro atom and which C₃₋₇-heterocycloalkyl-C₁₋₂-alkyl group contains one or two heteroatoms from the group (O, N, S), or R₂ represents a phenyl, benzyl, phenethyl or phenylpropylgroup which may be substituted on their phenyl ring with with 1-3 substituents Y, wherein Y has the abovementioned meaning, or R₂ represents a pyridyl, thienyl or naphtyl group, which napthyl group may be substituted with a halogen atom, a methyl group or a methoxy or trifluoromethyl group, with the proviso that when R₂ represents phenyl, R is not a 4-chlorophenyl group,
R₃ represents a hydrogen atom or a branched or linear C₁₋₃ alkyl group,
R₄ represents a branched or linear C₁₋₁₀ alkyl or C₃₋₈-cycloalkyl-C₁₋₂-alkyl group, branched or linear C₁₋₁₀ alkoxy, C₃₋₈ cycloalkyl, C₅₋₁₀ bicycloalkyl, C₅₋₁₀-bicycloalkyl-C₁₋₂-alkyl, C₆₋₁₀ tricycloalkyl, C₆₋₁₀ tricycloalkyl-methyl, branched or linear C₃₋₁₀ alkenyl, C₅₋₈ cycloalkenyl, which groups may contain one or more heteroatoms from the group (O, N, S) and which groups may be substituted with a hydroxy group, 1-3 methyl groups, an ethyl group or 1-3 fluoro atoms, or R₄ represents a phenyl, phenylamino, phenoxy, benzyl, phenethyl or phenylpropyl group, optionally substituted on their phenyl ring with 1-3 substituents Y, wherein Y has the abovementioned meaning, or R₄ represents a pyridyl or thienyl group, or R₄ represents a group NR₅R₆ wherein
R₅ and R₆—together with the nitrogen atom to which they are attached—form a saturated or unsaturated, monocyclic or bicyclic, heterocyclic group having 4 to 10 ring atoms, which heterocyclic group contains one or more heteroatoms from the group (O, N, S) and which heterocyclic group may be substituted with a branched or linear C₁₋₃ alkyl, phenyl, hydroxy or trifluoromethyl group or a fluoro atom, or
R₃ and R₄—together with the nitrogen atom to which they are attached—form a saturated or unsaturated, monocyclic or bicyclic, heterocyclic group having 4 to 10 ring atoms, which heterocyclic group contains one or more heteroatoms from the group (O, N, S) and which heterocyclic group may be substituted with a branched or linear C₁₋₃ alkyl, phenyl, amino, hydroxy, methoxy, cyano or trifluoromethyl group or a fluoro or chloro atom,
and pharmacologically acceptable salts thereof, as well as prodrugs, which are derivatives of the compounds having formula (I) wherein a group is present which is easily removed after administration, such as amidine, enamine, a Mannich base, a hydroxyl-methylene derivative, an O-(acyloxymethylene carbamate) derivative, carbamate or enaminone.

3. A compound as claimed in claim 2, or a salt thereof, for use as a medicament

4. Pharmaceutical compositions containing at least one compound as claimed in claim 2 as active ingredient.

5. Use of a compound as claimed in claim 2 for the preparation of a pharmaceutical composition for the treatment of disorders involving
cannabinoid neurotransmission such as psychosis, anxiety, depression, attention deficits, memory disorders, cognitive disorders, appetite disorders, obesity, addiction, appetence, drug dependence and neurological disorders such as neurodegenerative disorders, dementia, dystonia, muscle spasticity, tremor, epilepsy, multiple sclerosis, traumatic brain injury, stroke, Parkinson's disease, Alzheimer's disease, epilepsy, Huntington's disease, Tourette's syndrome, cerebral ischaemia, cerebral apoplexy, craniocerebral trauma, stroke, spinal cord injury, plaque sclerosis, viral encephalitis, demyelinisation related disorders and other diseases involving cannabinoid neurotransmission, including the treatment of septic shock, glaucoma, cancer, diabetes, emesis, nausea, asthma, respiratory diseases, gastrointestinal disorders, gastric ulcers, diarrhoea and cardiovascular disorders.

5. A compound of general formula (IV) 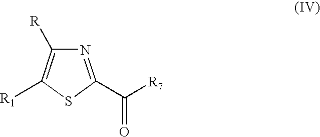
wherein one of R and R₁ represents a phenyl or 3-pyridinyl or 4-pyridinyl group, optionally substituted with 1-3 substituents Y, wherein Y has the abovementioned meaning and the other moiety represents a C₂₋₈ branched or linear alkyl group, C₃₋₈ branched or linear heteroalkyl group containing one heteroatom from the group (N, O, S) or an SO₂ group, C₃₋₇ cycloalkyl group, C₃₋₇-cycloalkyl-C₁₋₃-alkyl group, C₃₋₇-heterocycloalkyl-C₁₋₃-alkyl group which groups may be substituted with a hydroxy, methoxy, methyl, trifluoromethylsulfonyl or trifluoromethyl group or a fluoro atom and which C₃₋₇-heterocycloalkyl-C₁₋₃-alkyl group contains one or two heteroatoms from the group (O, N, S), or said other moiety represents a benzyl group optionally substituted on its phenyl ring with 1-3 substituents Y, wherein Y has the abovementioned meaning and R₇ represents a hydroxy, C₁₋₄ branched or linear alkoxy group or a benzyloxy group or a chloro atom or a N-methoxy-N-methylamino group, such compounds being useful in the synthesis of compounds of general formula (I).
